(19) United States
(12) Patent Application Publication
(10) Pub. No.: US 2008/0154222 A1
(43) Pub. Date: Jun. 26, 2008

(54) Catamenial and sanitary tampons

(76) Inventor: Bernard Chaffringeon, Daillens (CH)

Correspondence Address: OLIFF & BERRIDGE, PLC, P.O. BOX 320850, ALEXANDRIA, VA, 22320-4850, US

(21) Appl. No.: 11/984,880

(22) Filed: Nov. 23, 2007

Related U.S. Application Data
(60) Provisional application No. 60/860,798, filed on Nov. 24, 2006.
(60) Provisional application No. 60/880,023, filed on Jan. 12, 2007.
(60) Provisional application No. 60/898,710, filed on Feb. 1, 2007.
(60) Provisional application No. 60/920,776, filed on Mar. 30, 2007.
(60) Provisional application No. 60/907,411, filed on Apr. 2, 2007.
(60) Provisional application No. 60/929,490, filed on Jun. 29, 2007.

(51) Int. Cl. A61F 13/20; D04H 1/00

(52) U.S. Cl. 604/361; 604/379; 604/378; 604/385.18; 604/385.01; 28/120; 28/118

(57) ABSTRACT

In a tampon having a body of an absorbent material, the body has at least one zone in which expansion and/or absorption by the body of the tampon is limited.

### CROSS-REFERENCE TO RELATED APPLICATIONS

This Application claims the benefit of U.S. Provisional Application No. 60/860,798, filed Nov. 24, 2006; U.S. Provisional Application No. 60/880,023, filed Jan. 12, 2007; U.S. Provisional Application No. 60/898,710, filed Feb. 1, 2007; U.S. Provisional Application No. 60/920,776, filed Mar. 30, 2007; U.S. Provisional Application No. 60/907,411, filed Apr. 2, 2007; and U.S. Provisional Application No. 60/929,490, filed Jun. 29, 2007, the entire contents of each are hereby incorporated by reference.

### BACKGROUND

This disclosure is generally related to the field of catamenial and/or sanitary tampons and to devices permitting absorption of the body's natural waste fluids.

Many sanitary articles exist, especially for absorbing menstrual fluids, in particular catamenial and/or sanitary tampons made of absorbent material in the form of sheets and/or bands that are folded and/or rolled up, using techniques well known to persons skilled in the art, in order to form tampons that are inserted, with or without applicators, into the vaginas of the users.

However, although there are many models made of various materials and with absorption capacities that vary according to the particular requirements and losses, problems associated with leaking and/or soiling still exist that are due either to absorption capacities that are too low or to problems of volume, or even poor adherence to the vaginal wall, or a drainage phenomenon due in particular to the withdrawal devices, namely the threads and/or strings designed to remove the used tampon.

A problem also exists at the moment of withdrawal when the withdrawal devices, namely the threads and/or strings designed to remove the used tampon, and the vaginal wall can exert a pressure on the tampons and cause drying, which leads to a flow of fluid that soils the user's hands. Moreover, given that the lower part of the tampon, also called the proximal part, is greatly enlarged, this part has a tendency to cause discomfort or injuries by rubbing on the vaginal wall, especially in the area of muscular narrowing of the vaginal wall.

Many devices have already been proposed. For example, patent application WO 2006/094753 discloses a tampon with an impermeable zone formed by a plastic film, and zones of variable absorption in order to avoid drying of the vaginal walls. Moreover, U.S. Pat. No. 3,693,622 discloses tampons and other sanitary articles which, at their ends, comprise zones impregnated with fluid-repelling compositions.

The devices described in these documents nevertheless have numerous disadvantages, such as the possibility of the fluid-repelling compositions migrating into the whole of the tampon and/or the sanitary article after production, the possibility of the fluid-repelling compositions being transferred to the user's finger at the moment of insertion of the tampon, and especially the impossibility of limiting the drying effect during withdrawal. Moreover, the disclosed devices do not solve the problem of the shaping and adaptation of a tampon such that it better matches the shape of the vaginal walls.

U.S. Pat. No. 7,060,057 discloses tampons comprising nonabsorbent zones arranged on the outer surface of the tampon that permit increased comfort at the interface between the tampon and the vaginal walls.

U.S. Pat. No. 7,097,638 discloses tampons comprising zones that form reinforcements on the outer walls of the tampon in order to improve comfort while at the same time maintaining the absorption capacities of the tampons.

U.S. Pat. No. 3,965,905 discloses tampons formed of a plurality of absorbent product parts connected by a withdrawal cord.

These different devices, however, do not significantly improve absorption while at the same time improving comfort and retaining other important properties. The tampon described in U.S. Pat. No. 3,965,905, for example, does not provide a tampon that is sufficiently cohesive to permit its insertion without an applicator.

U.S. Patent Application Publication No. 2003/0097108 describes a tampon comprising an absorbent compressed body whose outer face is largely covered by an absorbent shroud that forms a skirt beyond the proximal end of the body of the tampon.

U.S. Patent Application Publication No. 2005/0096620 describes a tampon obtained by rolling up a layer of absorbent material, the layer having an edge intended to constitute the proximal end after rolling, and being covered by an absorbent shroud before rolling.

U.S. Pat. No. 4,211,225 describes a tampon permitting improved comfort during its withdrawal. For this purpose, a first shroud and then a second shroud are attached to the body of the tampon and are fixed in the area of the distal end of the tampon. The first shroud is designed such that the frictional forces generated during withdrawal between the body of the tampon and the first shroud are less than the frictional forces between the second shroud and the surrounding tissues.

### SUMMARY

The present disclosure relates to a catamenial or sanitary tampon with an absorbent body, where the tampon comprises means for limiting the expansion and/or the absorption of at least one zone of the body of the tampon when the tampon comes into contact with a fluid that is to be absorbed.

Thus, the control of the expansion and/or of the absorption of the body of the tampon in this zone permits formation, at this location, of a fluidic barrier that is more or less impervious with respect to the absorbed fluid. In some embodiments, the body of the tampon may comprise a plurality of zones formed by fluidic barriers.

In embodiments, the expansion and/or absorption are limited by a solid hydrophobic substance whose melting point is greater than 37° C., which partially or totally impregnates the absorbent material from which the tampon is made.

The disclosure is also directed to tampons having a withdrawal thread that is also at least partially impregnated with a solid hydrophobic substance whose melting point is greater than 37° C.

By using a solid hydrophobic substance whose melting point is greater than 37° C., the zones in which the expansion and/or the absorption are limited are permanent, that is to say, the zones are nondeformable or are only minimally deformable, even when the tampon is saturated.

A solid hydrophobic substance whose melting point is greater than 37° C. may have certain properties that are similar to those of beeswax, that is to say a substance whose plasticity permits malleability at room temperature, whose viscosity is low when the substance is melted, and that is totally hydrophobic.

In embodiments, the hydrophobic substance is chosen from the group consisting of hydrogenated and/or nonhydrogenated petrolatum, paraffin and/or stearin. In embodiment variant, the hydrophobic substance is hydrogenated petrolatum.

In embodiments, the hydrophobic substance is chosen from the group consisting of hydrogenated and/or nonhydrogenated oils and/or fats.

In embodiments, the hydrophobic substance is chosen from the group consisting of natural and/or synthetic waxes, for example carnauba wax, jojoba oil, hard paraffin and so-called microcrystalline wax (waxes extracted from petroleum), and silicone waxes (waxes obtained by synthesis). In embodiments, the hydrophobic substance is beeswax. Beeswax is principally composed of an ester of ethylene glycol and two fatty acids. In other embodiments, the hydrophobic substance is carnauba wax.

The disclosure is also directed to a tampon configured so that the part in which the expansion and/or the absorption are limited is situated at the proximal end of the tampon.

In embodiments, the part in which the expansion and/or the absorption are limited is situated at a distance of more than 0.5 mm from the proximal end of the tampon.

In embodiments, the zone in which the expansion and/or the absorption is limited forms a spiral inside and outside the tampon.

In embodiments, the tampon additionally comprises another zone in which the expansion and/or the absorption are limited, situated half way between the two ends of the tampon.

In embodiments, the tampon additionally comprises another part in which the expansion and/or the absorption are limited, forming a spiral outside the tampon. In such embodiments, and in contrast to the tampon described and shown in EP 1 383 453, the spiral shape obtained may be nondeformable or only minimally deformable, even after saturation of the tampon with a hydrophilic substance, and this spiral shape makes it possible to channel the flow and thus avoid flow of fluid along the vaginal walls, and also to increase the time of contact between the absorbent material and the flow, thus permitting improved absorption and a greater capacity of the tampon.

The tampons may be impregnated with the solid hydrophobic substance whose melting point is greater than 37° C. in the melted state and before and/or after formation of the tampon. When the substance is applied before formation of the tampon, it is applied to the band before folding and/or compression and/or rolling of the band.

A zone formed by a line measuring several tenths of a millimeter to several millimeters is applied continuously or discontinuously to the front face and/or rear face of the band, optionally in several zones, and optionally nonsymmetrically. The zones of impregnation may be arranged on what will become the lower part, middle part, and/or the upper part of the tampon in order to create one or more reservoirs with these successive barriers.

The zones may be arranged in such a way that, after folding and/or compression of the band, a spiral shape is obtained that is nondeformable or only minimally deformable, even after saturation of the tampon with a hydrophilic substance. This spiral shape makes it possible to channel the flow, and thus avoid flow of fluid along the vaginal walls, and also to increase the time of contact between the absorbent material and the flow, thus improving absorption and capacity of the tampon.

In embodiments, the tampon comprises an impregnation zone in the form of an upturned funnel which, by diverting the flow from the center of the tampon, makes it possible to avoid accumulation of fluid at the site of fixation of a withdrawal thread and to avoid leaks due to the presence of this withdrawal thread.

In embodiments, the means for limiting the expansion and/or the absorption are formed by at least one restriction member encircling the corresponding zone of the body of the tampon, while allowing the zone to be maintained in its initial shape when the body of the tampon inflates under the effect of the absorption.

In embodiments, the restriction member may be made in the form of a ring, a washer, a string, a collar, or one or more foldable restrictor bands made of permeable material.

In embodiments, the restriction member comprises, on its outer face, at least one smooth zone that makes it possible to limit the friction during removal of the tampon, so as to avoid any injury.

In embodiments, the restriction member has a spiral shape either on the distal part of the tampon or on the proximal part of the tampon.

Advantageously, the ring is formed with the protector of the tampon.

In embodiments, each restrictor band comprises at least one orifice near each of its two ends, the orifice having a diameter that is substantially equal to that of the compressed tampon. Where appropriate, the restrictor band may comprise at least a third orifice in the central part for passage of the withdrawal thread.

In embodiments, the tampon comprises at least two superposed restrictor bands arranged perpendicular to each other before folding.

In embodiments, the restrictor band is designed to have a length that ensures the restrictor band does not disturb the natural development of the geometry of the tampon body when the tampon is placed in contact with a fluid that is to be absorbed.

The disclosure also relates to a strip of absorbent material comprising zones in which the absorbent material is partially or totally impregnated with a solid hydrophobic substance whose melting point is greater than 37° C.

The disclosure is also directed to a method for producing a tampon, wherein the method finally comprises, after a step of folding and/or of compression and/or of impregnation of an absorbent material such as an absorbent strip including a hydrophobic substance, a step of heating to a temperature above the melting point of the hydrophobic substance, making it possible to bind the impregnated zones.

The disclosure is also directed to a tampon comprising a saturation indicator at its proximal end. In embodiments, the indicator is formed by the proximal zone situated at the proximal end of the tampon comprising at least one zone in which the expansion and/or the absorption are limited, the zone being formed by partial or total impregnation of the absorbent material of the tampon with a solid hydrophobic substance whose melting point is greater than 37° C. In embodiments, the indicator is formed by the proximal zone situated at the proximal end of the tampon comprising at least one zone in which the expansion and/or the absorption are limited, the zone being formed by placement of a restriction member, for example in the form of a ring, string, collar or restrictor band(s) encircling the tampon and allowing it to be maintained in its initial shape in the zone in question, when the tampon comes to inflate under the effect of the absorption. The various embodiments may be separate or in combination with one or more of the other embodiments.

### BRIEF DESCRIPTION OF THE DRAWINGS

Embodiments will be better understood from the detailed description given below and by reference to the attached drawings, in which:

FIGS. 1 and 2 are diagrammatic views of two strips of absorbent material;

FIGS. 3 and 3a are diagrammatic views of two other strips of absorbent material during the injection of the hydrophobic substance;

FIG. 4 is a diagrammatic view of another strip of absorbent material when laid out flat;

FIGS. 5 and 5a are diagrammatic views, in longitudinal section, of a tampon according to the embodiment of FIG. 16 before and after heating of the hydrophobic substance;

FIG. 6 is a diagrammatic view of the machine for producing a tampon according to the embodiment of FIG. 16;

FIGS. 7 and 8 are diagrammatic views of a first strip of absorbent material and of a second strip of absorbent material, respectively, when laid out flat;

FIGS. 7a and 8a are perspective views of the tampons obtained respectively from the strips of absorbent material shown in FIGS. 7 and 8;

FIG. 9 is a diagrammatic view of a third strip of absorbent material;

FIGS. 10, 11 and 12 are diagrammatic views of a fourth strip of absorbent material, a fifth strip of absorbent material and a sixth strip of absorbent material, respectively, when laid out flat;

FIGS. 10a, 11a and 12a are perspective views of the tampons obtained respectively from the strip of absorbent material shown in FIGS. 10, 11 and 12;

FIG. 13 is a diagrammatic view of another strip of absorbent material when laid out flat;

FIG. 13a is a perspective view of the tampon obtained from the strip of absorbent material shown in FIG. 13;

FIG. 14 is a diagrammatic view of four other illustrative embodiments of a strip of absorbent material;

FIGS. 15 and 15a are diagrammatic perspective views of two tampons according to the embodiment of FIG. 16 during the phase of injection of the hydrophobic substance;

FIGS. 16 and 16a are diagrammatic perspective views of a tampon according to an embodiment, before use (FIG. 16) and after use (FIG. 16a);

FIG. 17 is a diagrammatic view of a traditional tampon and FIG. 17a is a diagrammatic view of a tampon according to the embodiment of FIG. 16 during the withdrawal phase;

FIG. 18 is a diagrammatic view of a tampon according to another embodiment;

FIG. 18a is a diagrammatic view of a tampon according to another embodiment;

FIGS. 19 and 19a are partial diagrammatic views, in longitudinal section, of two separate rings with which a tampon according to the embodiment of FIG. 18 is equipped;

FIG. 20 is a diagrammatic end view of a restriction member in the form of one or more washers;

FIGS. 20a and 20b are diagrammatic perspective views of another tampon equipped with restriction members shown in FIG. 20, before and after use;

FIG. 21 is a diagrammatic end view of a restriction member in the form of another type of washer;

FIG. 21a is a diagrammatic perspective view of another tampon equipped with a restriction member shown in FIG. 21;

FIG. 22 is a diagrammatic end view of a restriction member in the form of another type of washer;

FIG. 22a is a diagrammatic perspective view of another tampon equipped with a restriction member shown in FIG. 22;

FIG. 22b is a diagrammatic perspective view of the withdrawal thread with which the tampon shown in FIG. 22a is equipped;

FIGS. 23 and 23a are diagrammatic views of a restrictor band forming a restriction member, before folding (FIG. 23) and after folding (FIG. 23a);

FIG. 24 is a diagrammatic flat view of another restrictor band forming a restriction member;

FIG. 24a is a diagrammatic perspective view of another tampon equipped with the restrictor band shown in FIG. 24;

FIG. 25 is a diagrammatic flat view of a restrictor band forming a restriction member;

FIG. 25a is a diagrammatic view, in longitudinal section, of another restrictor band forming a restriction member;

FIGS. 25b and 25c are diagrammatic perspective views of another tampon equipped with the restrictor band shown in FIG. 25, before and after use;

FIG. 26 is a diagrammatic perspective view of another restrictor band forming a restriction member, after folding;

FIG. 27 is a diagrammatic view of the placement of the restriction member, shown in FIG. 23a, around a tampon;

FIG. 27a is a diagrammatic perspective view of the tampon shown in FIG. 27, once the restriction member is in place;

FIG. 27b is a view similar to FIG. 27a, after use;

FIG. 28 is a diagrammatic perspective view of a tampon as shown in FIG. 27a, additionally comprising a fluidic barrier formed with the aid of a hydrophobic substance;

FIG. 29 is a diagrammatic end view of an orifice according to another embodiment;

FIGS. 30 and 31 are diagrammatic views of two other restriction members that are cross-shaped; and

FIG. 32 is a diagrammatic perspective view of a tampon with a saturation indicator.

### DETAILED DESCRIPTION OF EMBODIMENTS

In the following detailed description of the figures defined above, the same elements, or the elements performing identical functions, will keep the same reference numbers, so as to make various embodiments easier to understand. For example, reference number 1 is given to any tampon according to one embodiment, and reference number 10 is given to any tampon according to a second embodiment of the invention.

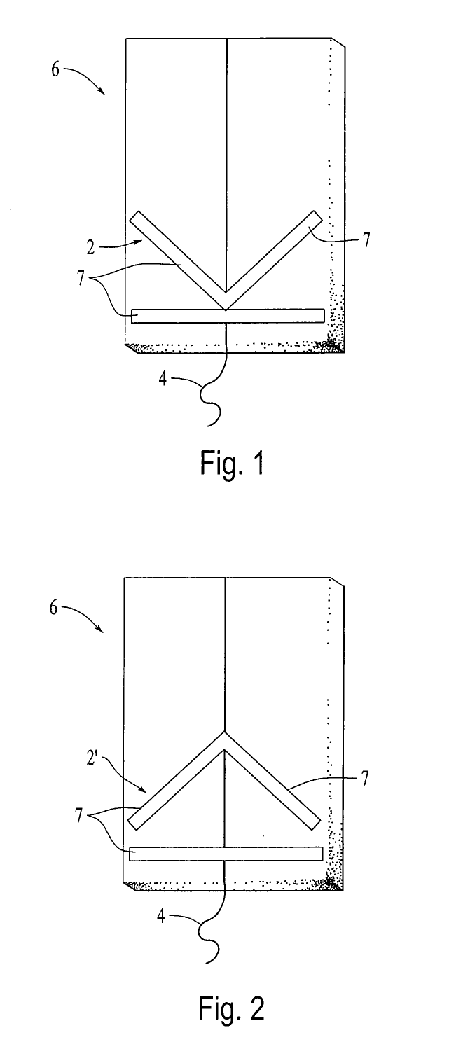

Tampons 1 are obtained from strip of absorbent material 6, such as are shown in FIGS. 1 and 2.

The form of the impregnation zone is specially designed as a function of the desired result.

In FIG. 1, the impregnation zone forms a funnel 2 which channels the flow toward the proximal end of the tampon 1 and, for example, avoids the peripheral leaks due to the flow passing to the zone of contact between the walls (not shown) and the tampon 1.

In FIG. 2, the impregnation zone is an upturned funnel 2′ which, by diverting the flow from the center of the tampon 1, avoids accumulation of fluid at the site of fixation of the withdrawal thread 4 and avoids leaks due to the presence of the withdrawal thread 4.

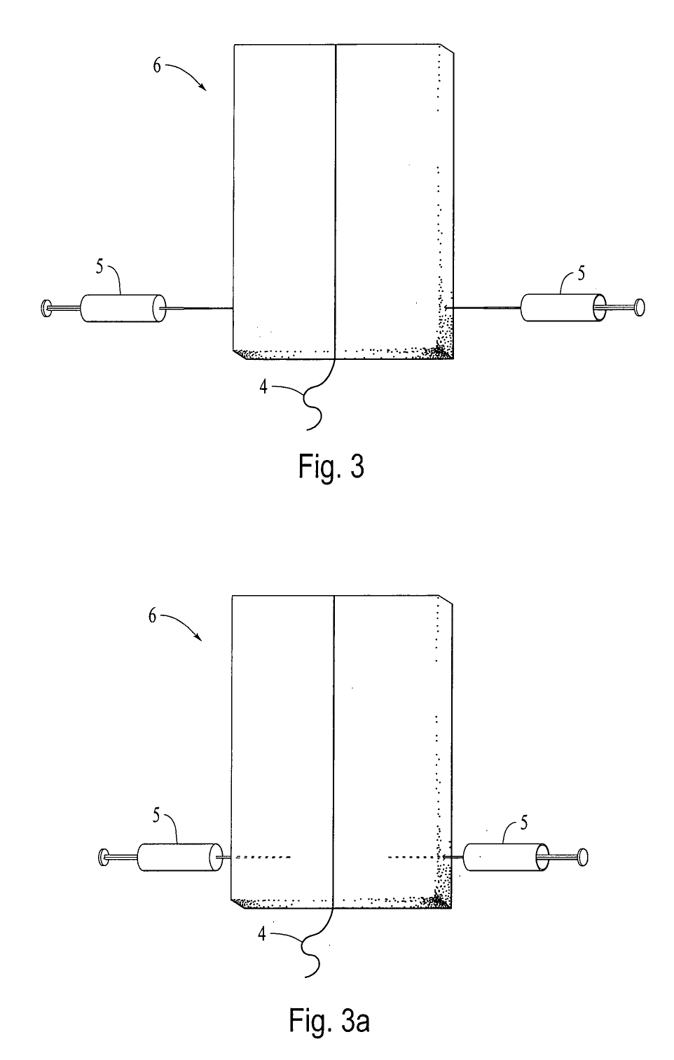

FIGS. 3 and 3a show examples of injectors 5 for injecting a hydrophobic substance 7 into the thickness of a strip of absorbent material 6, the injection being carried out from the side.

After formation of the tampon 1, the zones of impregnation may be heated to temperatures above the melting point of the hydrophobic substance 7, so as to cause the hydrophobic substance 7 to melt and to better connect or affix the various layers that were saturated.

FIG. 4 shows a strip of absorbent material 6 that will form a tampon 1 by means of folding or compression. The hydrophobic substance 7 is applied in the transverse direction on one face, or on both faces and, if appropriate, within the thickness of the strip of absorbent material 6.

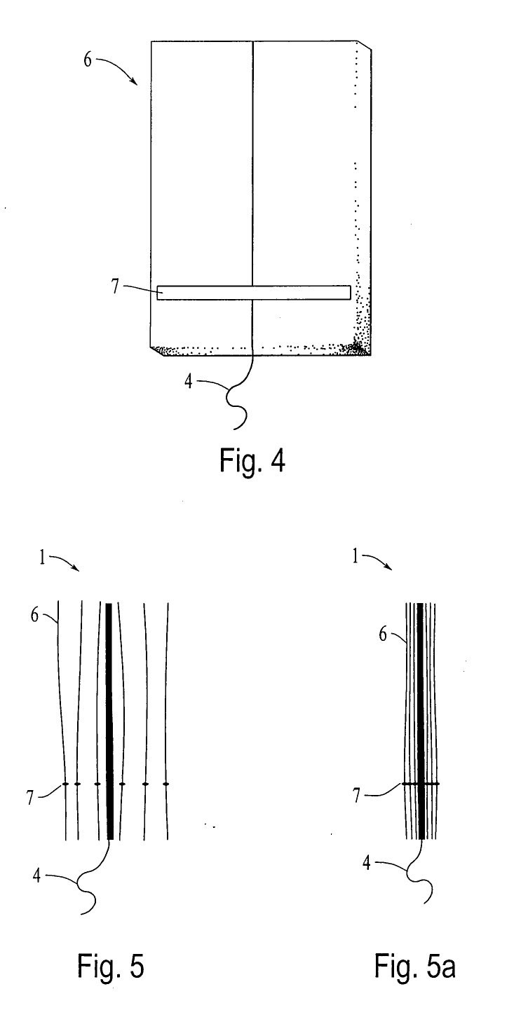

FIGS. 5 and 5a show a schematic view of a folded tampon 1, with the points of application of the hydrophobic substance 7. Depending on the stage of formation of the tampon 1, the points of hydrophobic substance 7 may be joined to one another, for example by thermofusion at the time of folding, rolling and/or packaging, or may remain free.

The spacing, and hence the barrier and/or filter effect, is quantified by the intensity of the heating and also by the thickness of the impregnation and, consequently, by the quantity of hydrophobic substance 7 applied.

Thus, the parts that are not in direct contact with this strong heat source will be joined to a lesser extent to one another. The effect of this is to create passages that will slow the downward movement of the blood and will thus make it possible to increase the absorption time and capacity of the tampon 1 by an effect of filtration and deceleration of the flow, thereby making it possible to increase the time of contact between the flow and the absorbent material from which the tampon is made.

Moreover, it must be clearly understood that the dilation and/or expansion of the proximal part is able to be controlled in terms of time. The dilation and/or expansion is able to be produced with a delay effect, or may even be rendered impossible, depending on the quantity of the hydrophobic substance, the temperature of the latter, and the temperature during the step of thermofusion used in its production.

Methods for the production of a tampon 1 may further comprise, after a step of folding, and/or of compression and/or of impregnation, a heating step that makes it possible to bind the impregnated zones.

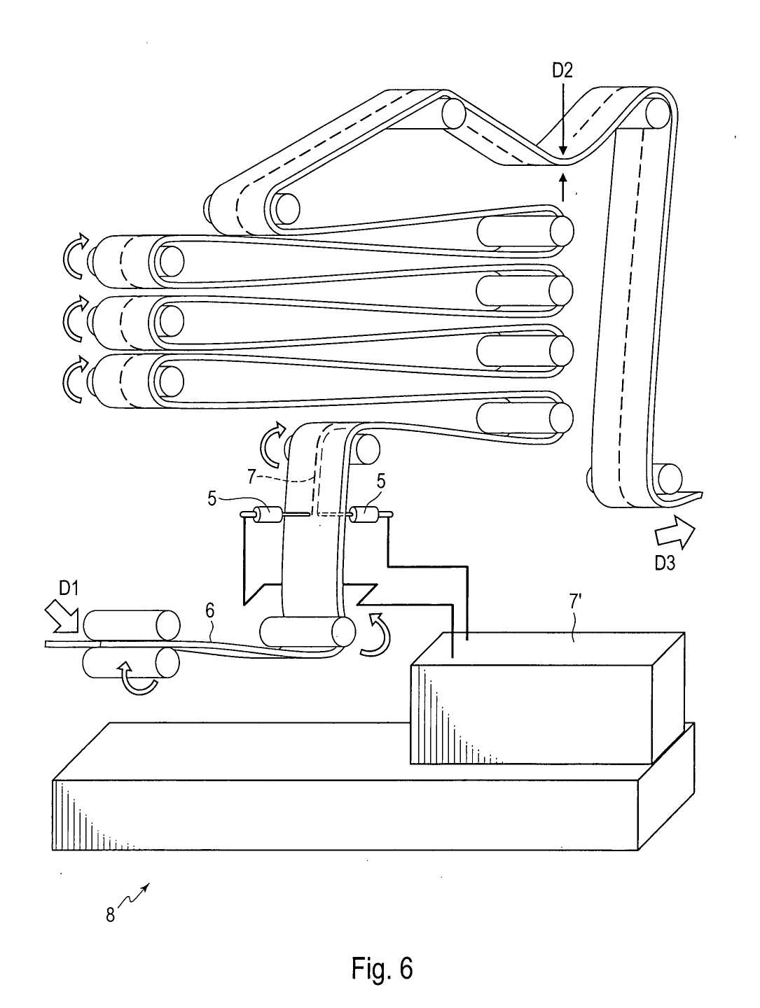

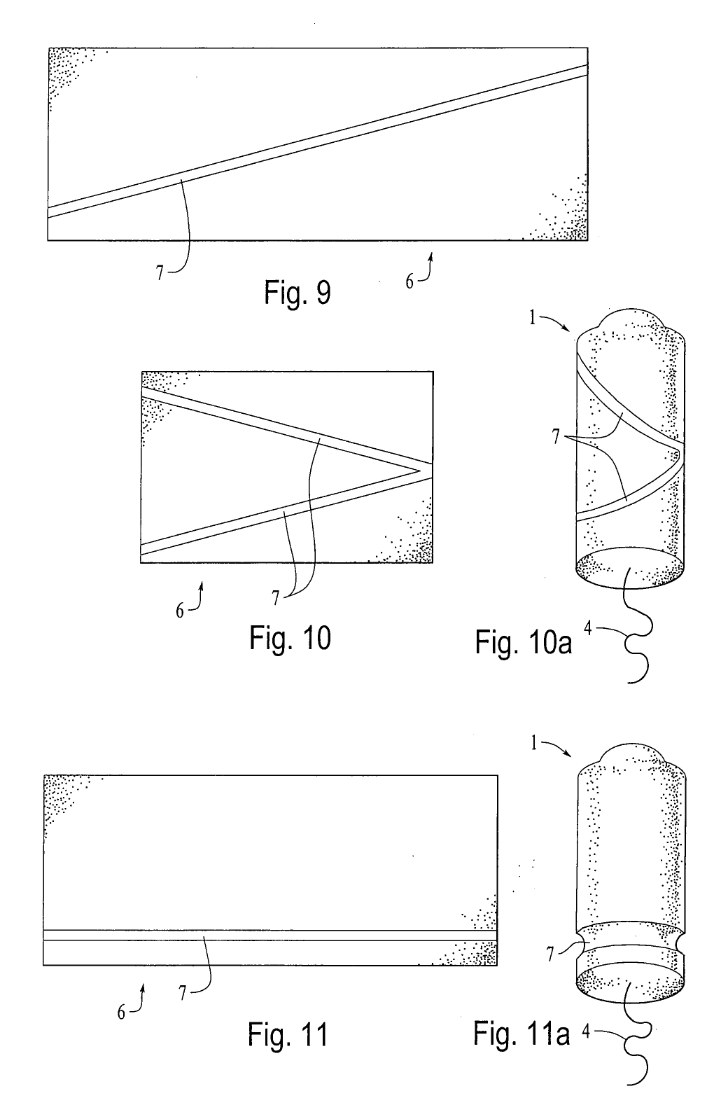

A tampon 1 may be produced using a machine 8 such as that shown schematically in FIG. 6. More particularly, this machine 8 serves as a support for injecting the strip of absorbent material 6 with hydrophobic substance 7 on one of the two faces, or both of them, or within the thickness of the strip of absorbent material, with the aid of the injectors 5 that are connected to an injection device 7′ for the hydrophobic substance 7. These injectors 5 are either single or multi-directional. For injections into the thickness of the strip of absorbent material 6, the injectors 5 are equipped with needles that penetrate the strip of absorbent material 6, as illustrated in FIGS. 3 and 3a. The injectors 5 may be of the traditional pump type, which may be mounted on an “XY” table to configure the forms. The injectors 5 are optionally mobile, in order to apply the hydrophobic substance 7 by creating forms such as those illustrated in FIGS. 1, 2, 7-10, and 13.

The path traveled by the strip of absorbent material 6 from its arrival at D1 to the outlet D3 makes it possible to lengthen the time between the application of the hydrophobic substance 7 and the zone of assembly of the tampon 1, and thus permits stiffening of the hydrophobic substance 7. In embodiments, a jet of air may be applied to accelerate this stiffening. One observes at D2 a flexion in the strip of absorbent material 6 to control the traction of the strip of absorbent material 6, and at D3 the exit of the strip of absorbent material 6 for entry into the following assembly machine.

The hydrophobic substance 7 may also be placed in a very precise manner such that it is located only inside the tampon 1 at the time the latter is rolled up or folded, and not on the visible outer part of the tampon 1. The barrier is located inside the tampon 1, and the first layer will permit absorption of the lateral leaks flowing along the wall.

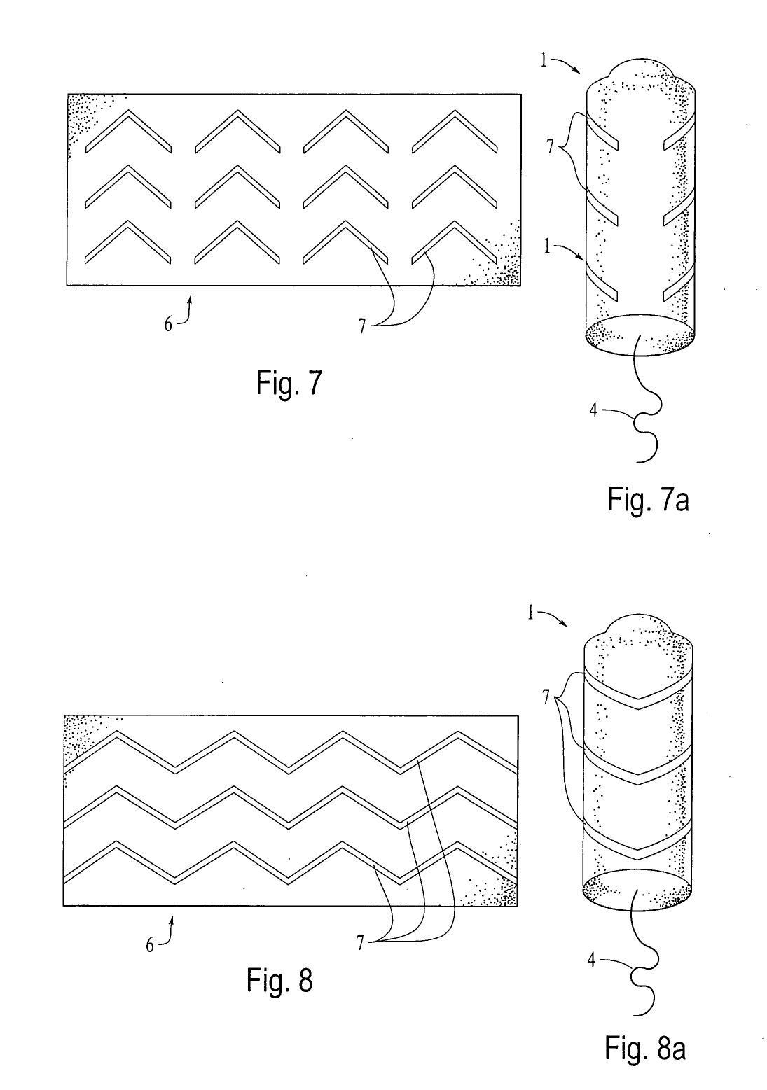

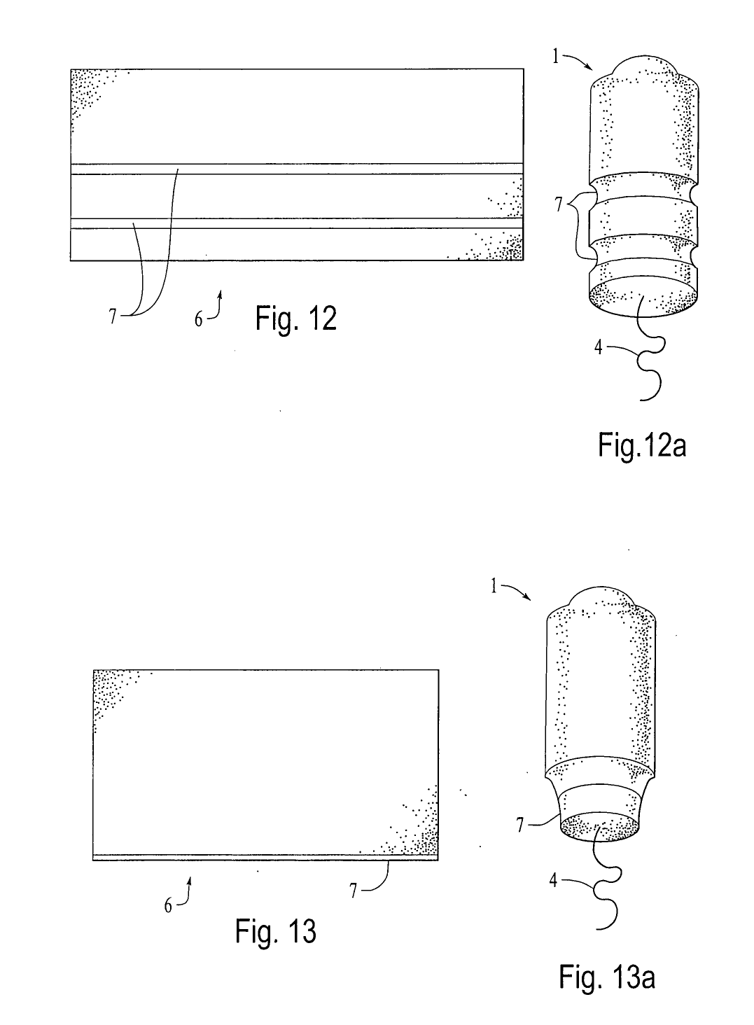

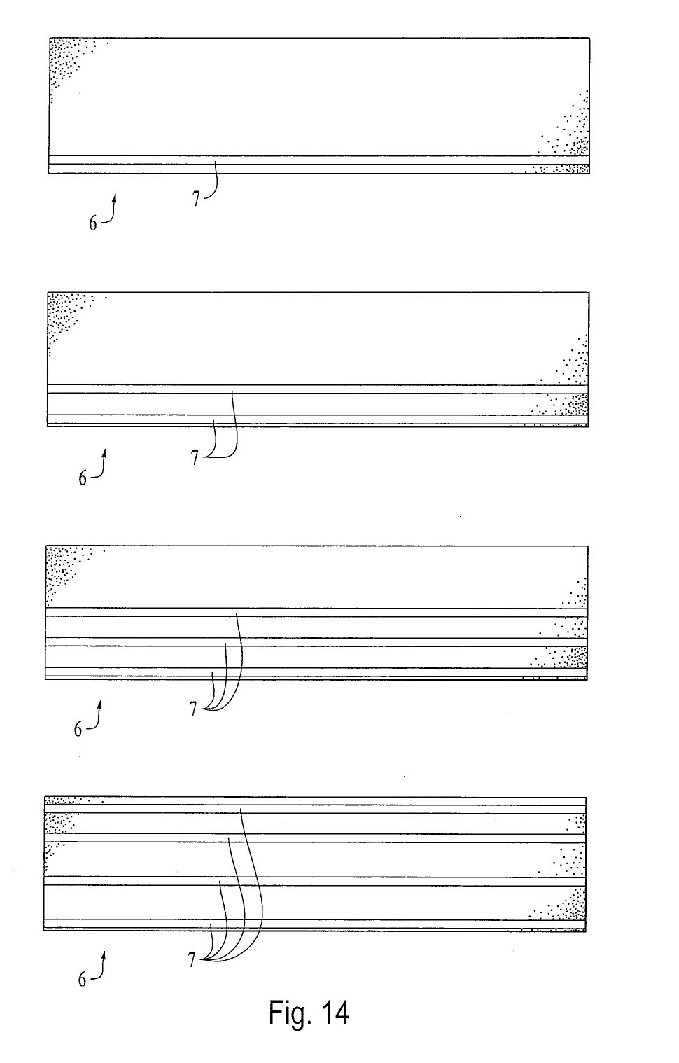

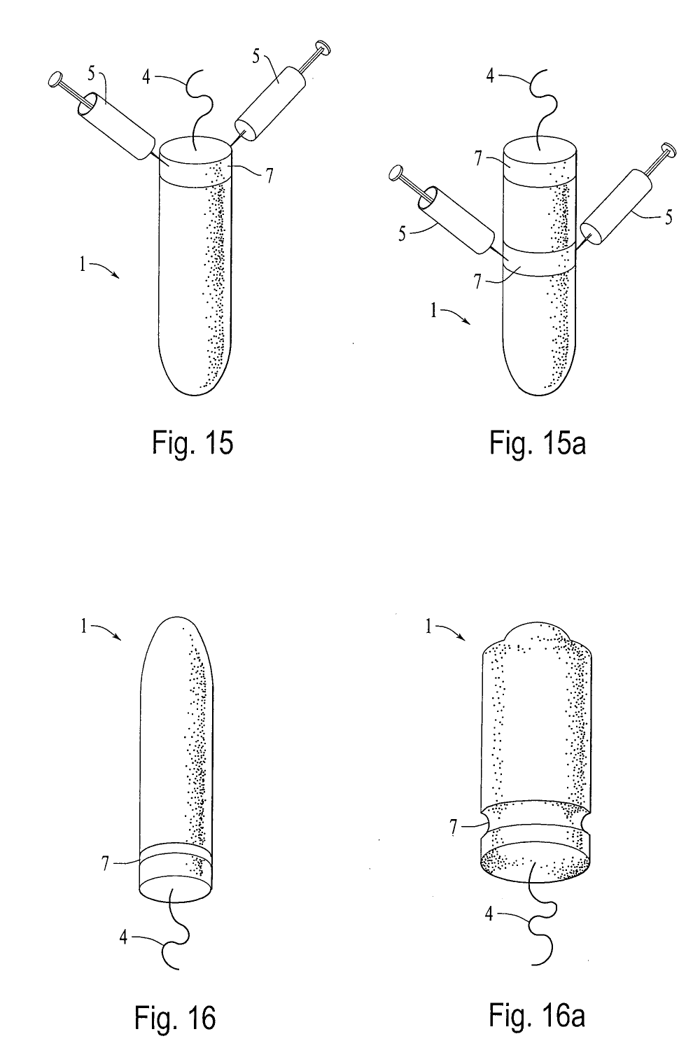

Different application variants are illustrated in FIGS. 7 to 12. FIGS. 13 to 15a schematically illustrate the results obtained and the methods of impregnation.

The application may be carried out on the tampon 1 when already formed, the melted hydrophobic substance 7 thus being applied with an injection or impregnation system, after protection of the zones that are not to be impregnated on the tampon 1 already formed.

It is also possible to soak the withdrawal thread 4 in the operating mode, either before being assembled or during the phase with the tampon 1.

A strip of absorbent material 6 of absorbent material may comprise zones in which the absorbent material is partially or totally impregnated with a solid hydrophobic substance 7 whose melting point is greater than 37° C. For example, the hydrophobic substances described herein may have a melting point equal to or greater than 38° C., 39° C., 40° C., 41° C., 42° C., 43° C., 44° C., 45° C., or 46° C. In embodiments, the hydrophobic substance has a melting point equal to or greater than 47° C., 49° C., 51° C., 53° C., 55° C., 57° C., 59° C., 61° C., or 63° C. In embodiments, the hydrophobic substance has a melting point equal to or greater than 68° C., 69° C., 70° C., 71° C., 72° C., 73° C., 74° C., 75° C., or 76° C.

The hydrophobic substances preferably do not dissolve in bodily fluids and preferably have a melting point that is higher than the internal temperature of the human body (37° C.) so that the hydrophobic substance remains solid while the tampon is in a vagina. Preferably, the hydrophobic substance has a melting point that is 45° C. or higher so that the hydrophobic substance remains solid during shipping and/or storage of the tampons where the tampons may be exposed to temperatures greater or significantly greater than 37° C. However, it is preferred that the melting point of the hydrophobic substance does not exceed 120° C., and more preferably does not exceed 100° C., thus making it more economical to manufacture the tampons in terms of costs and time. The lower the melting temperature of the hydrophobic substance, the less energy and time required to melt the hydrophobic substance for impregnation of the absorbent material and to allow or cause the impregnated hydrophobic substance to solidify.

In embodiments, the hydrophobic substance 7 impregnated on the strip of absorbent material 6 is chosen from the group consisting of hydrogenated and/or nonhydrogenated oils and/or fats.

In embodiments, the hydrophobic substance 7 impregnated on the strip of absorbent material 6 is chosen from the group consisting of natural and/or synthetic waxes.

In embodiments, the hydrophobic substance 7 impregnated on the strip of absorbent material 6 is hydrogenated petrolatum.

In embodiments, the hydrophobic substance 7 impregnated on the strip of absorbent material 6 is beeswax.

In embodiments, the hydrophobic substance 7 impregnated on the strip of absorbent material 6 is carnauba wax.

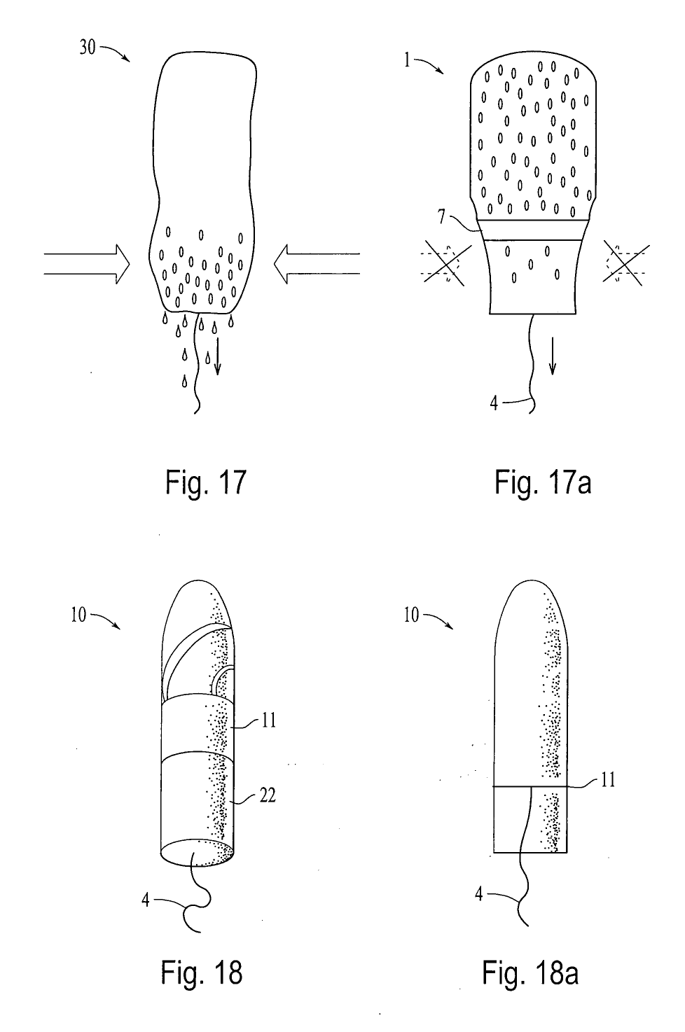

The mode of functioning of the absorption of the tampon 1 is illustrated in FIGS. 16, 16a and 17a. The absorption is improved by the barrier effect provided by the impregnation with the hydrophobic substance 7. This therefore makes it possible to reduce the first leaks. The zones that are impregnated are nondeformable or are minimally deformable, and they are not expanded, or are only minimally expanded, by the absorption of the flow of fluid. Moreover, such a tampon 1 may be designed to have an improved fluid absorption capacity of up to about 60%.

As is shown more specifically in FIG. 17, a traditional tampon 30 will tend to allow an untimely quantity of blood to escape via the proximal end and the withdrawal thread, on account of pressure exerted on the tampon by the muscles near the vaginal opening during removal of the tampon 30. This pressure is represented schematically by the white arrows in FIG. 17. By contrast, a tampon 1 configured with a fluidic barrier formed by a hydrophobic substance 7 as disclosed herein and as shown in FIG. 17a, counteracts this phenomenon by reducing the amount of blood that flows into the proximal area of the tampon during withdrawal of the tampon.

When the impregnation is partial and/or the tampon 1 does not undergo heating after formation of the tampon 1 with preimpregnated strip of absorbent material 6, and/or after the impregnation, the barrier formed simply has a filtering effect and makes it possible to increase the absorption by increasing the contact time between the flow and the absorbent material from which the tampon 1 is made.

According to a second embodiment of a tampon 10, the means for limiting the expansion and/or the absorption are formed by at least one restriction member 11 encircling the corresponding zone of the body and allowing the zone to be maintained in its initial shape when the body comes to inflate under the effect of the absorption. This restriction member 11 may, for example, be in the form of a ring, a string or a collar encircling the body of the tampon 10, as is illustrated in FIG. 18.

In an embodiment, the zone that is compressed or whose expansion and/or absorption are limited, functions as a means of reducing the passage of the fluid. The zone has a spiral shape either on the distal part of the tampon 10 or on the proximal part of the tampon 10.

In another embodiment, the restriction member 11 may be formed with a string, where at least a portion of the string is wrapped, tied, fastened, or otherwise affixed around the body of the tampon. As shown schematically in FIG. 18a, a withdrawal thread 4 may also be connected to and/or function as the restriction member 11.

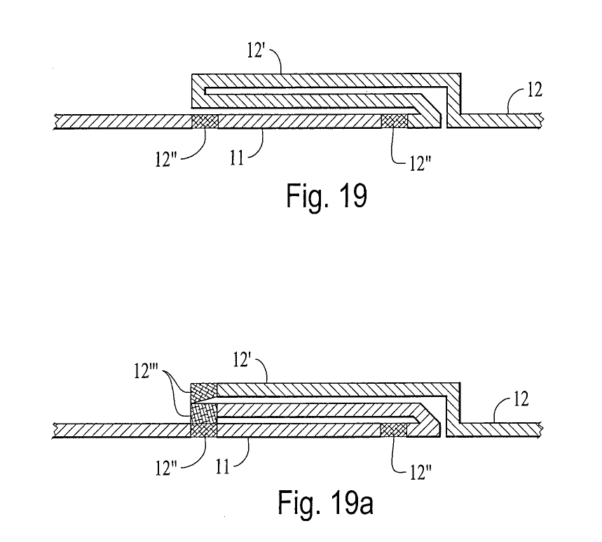

In another embodiment, the ring forming the restriction member 11 may be formed with the protector 12 or packaging of the tampon 10, such as a wrapper, which is cut or pre-slit or breakable so that the ring is separable from the protector 12 or packaging of the tampon 10, as is illustrated in FIGS. 19 and 19a.

This protector 12 has a tab 12′ for guaranteeing the protection of the ring 11 intended to remain in place after the insertion. During use of the tampon 10, the user takes hold of this tab 12′ in order to fragment the protector 12 at the area of two weld points or bonding points 12″. The ring 11 will then be formed by the remaining part of the protector 12 around the tampon 10.

Moreover, and as is shown in FIG. 19a, the tab 12′ may be reattached to the protector 12 via weld points or bonding points 12′″. This variant is particularly advantageous from the point of view of hygiene, because the ring 11 is free of any prior contact with the exterior before detachment of the protector 12.

The position of the ring 11 may be centered with respect to the distal and/or proximal ends and/or offset toward one of the ends.

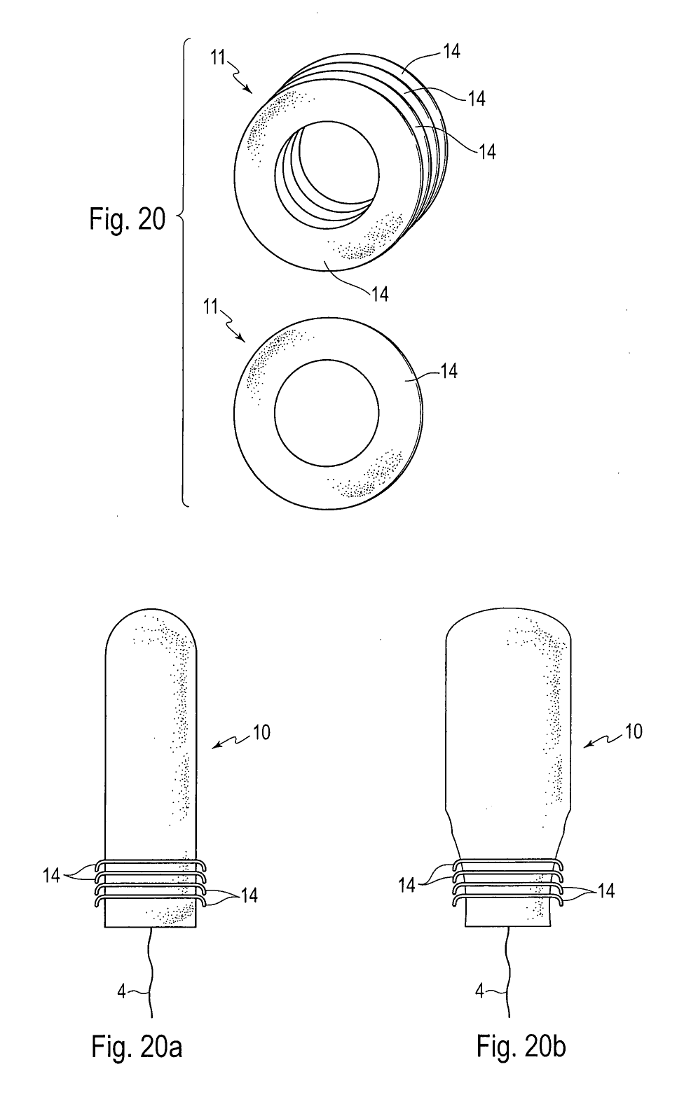

As is shown in FIGS. 20 and 20a, the ring may be formed by a single washer 14 or by a plurality of stacked washers 14, such as 2, 3, 4, 5, 6, 7, 8, 9, or 10 or more washers. The orifice of each washer 14 has a diameter substantially equal to that of the compressed body of the tampon 10. FIGS. 20a and 20b show a ring having four stacked washers 14.

As is shown in FIG. 21, the orifice of each washer 14 is preferably formed by making two slits 14′ perpendicular to each other, the effect of which is to delimit four substantially triangular flaps 14″ which are oriented upward or downward depending on whether the washer 14 is introduced via the top or bottom of the tampon 10. However, the orifice may be formed by two slits that are not perpendicular to each other, or by more than two slits. In FIG. 21a, the washer 14 has been slid on from the bottom of the tampon 10. The washers 14 may be secured to the tampon by affixing one or more of the triangular flaps 14″ to the tampon by hot pressing, hot melting, sewing, or any other method, as schematically depicted by the dashed lines in FIG. 21a.

Moreover, as is shown in FIG. 22, two opposite flaps 14″ may each comprise a hole 14′″ in such a way as to allow the passage of the withdrawal thread 4 shown in FIG. 22b. In FIG. 22a, the washer 14 thus formed has been slid on from the top of the tampon 10.

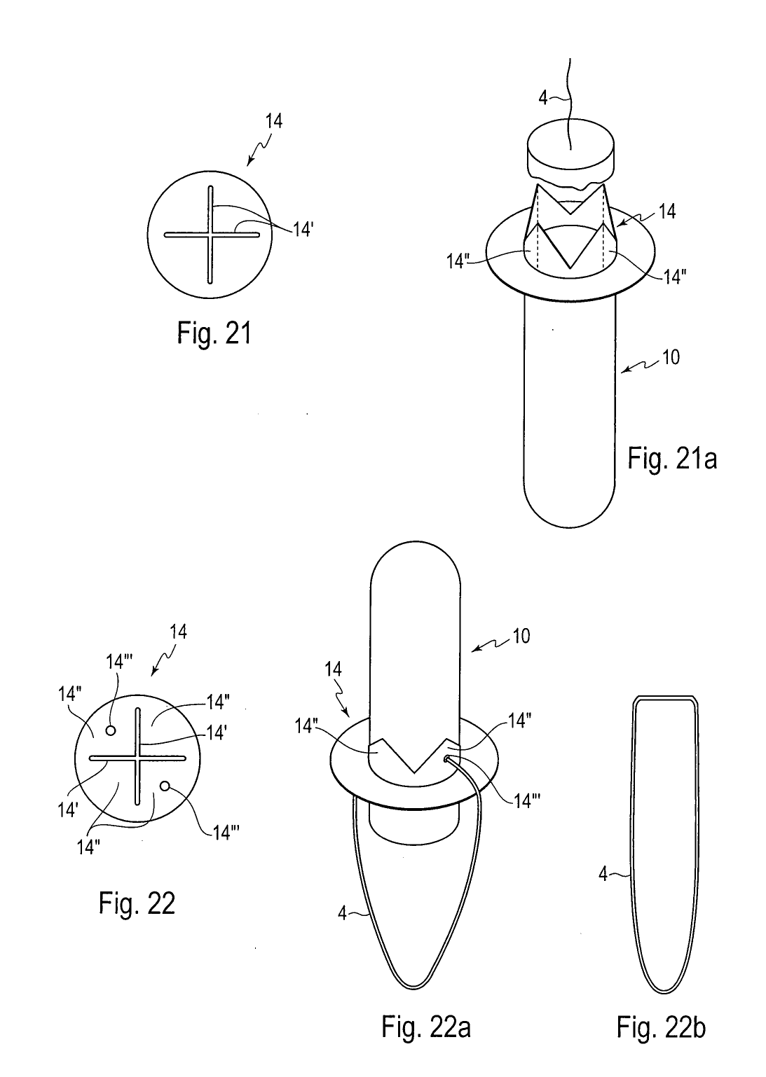

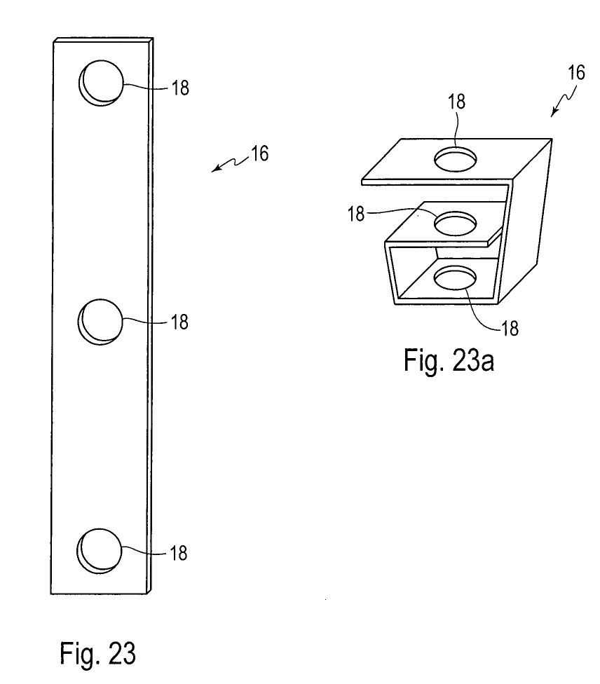

Referring to FIGS. 23 to 31, it is noted that each restriction member may be made of a permeable material in the form of a restrictor band 16 or in the form of a cross 17 prior to folding.

More precisely, a restrictor band 16 for the upper part of the tampon 10 has at least one nondeformable or minimally deformable orifice 18 near each of its two ends, and the diameter of this orifice 18 is chosen such as to be equal to that of the body of the compressed tampon 10. Thus, once the tampon 10 is in place, the body of the tampon will inflate under the effect of the absorption, while the zone of the body of the tampon encircled by the edge of the orifices 18 will remain compressed. These orifices 18 may therefore actually play the role of a restriction member, prohibiting the tampon 10 from changing diameter in the desired zones.

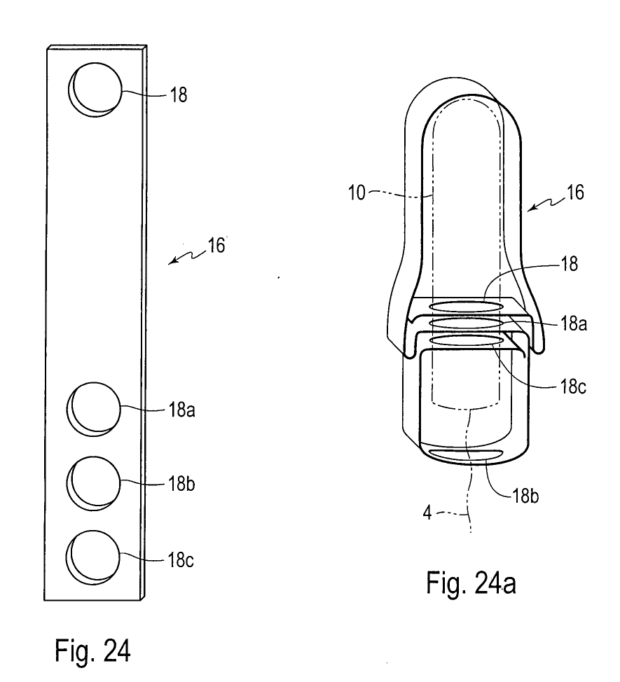

As is shown more specifically in FIGS. 23 and 24, the restrictor band 16 has a supplementary central orifice 18 when it is a restrictor band intended for the lower part of the tampon 10 and requiring passage of the withdrawal thread 4.

It must also be clearly understood that the same restrictor band 16 may be used for the lower part and the upper part of the tampon 10, as is shown in FIGS. 24 and 24a. The restrictor band 16 in this case comprise four orifices 18, 18a, 18b, and 18c.

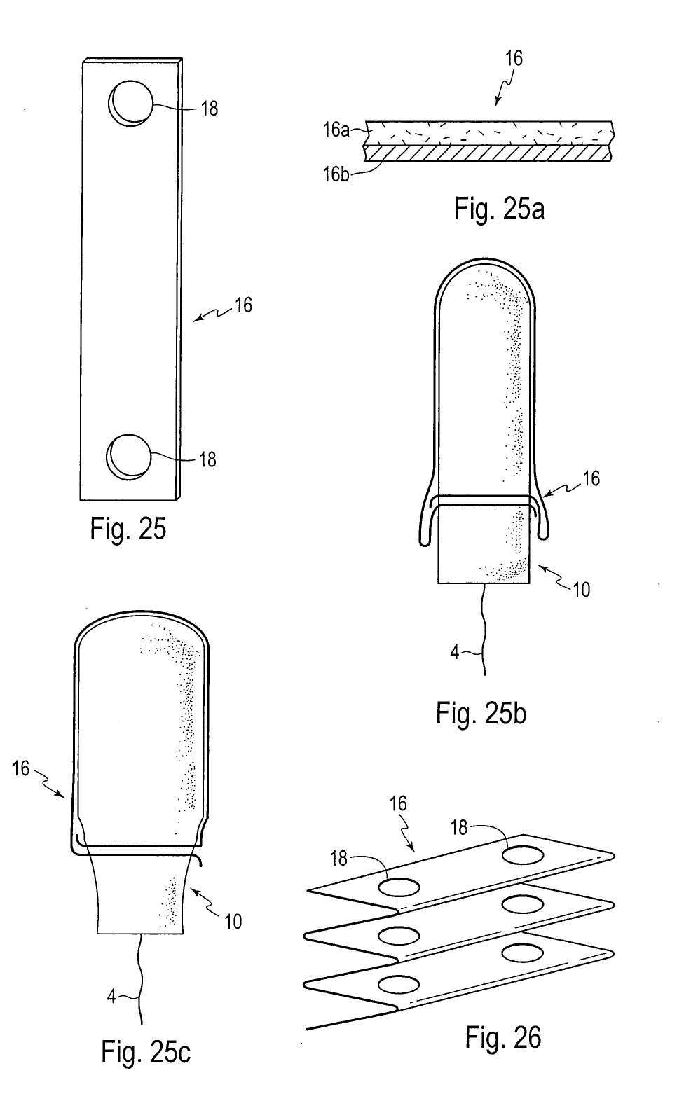

By contrast, the tampon 10 shown in FIG. 25b comprises a restrictor band 16, illustrated in FIG. 25, that has been folded and then inserted exclusively via the upper part of the tampon 10. FIG. 25c shows the tampon 10 after use.

Another restrictor band 16 according to embodiments, as shown in FIG. 25a, differs from the one described above in that it comprises, on the one hand, an inner absorbent layer 16a and, on the other hand, an outer impermeable layer 16b connected to the inner layer 16a.

As is shown in FIG. 26, the restrictor band 16 may be formed by folding in an accordion configuration.

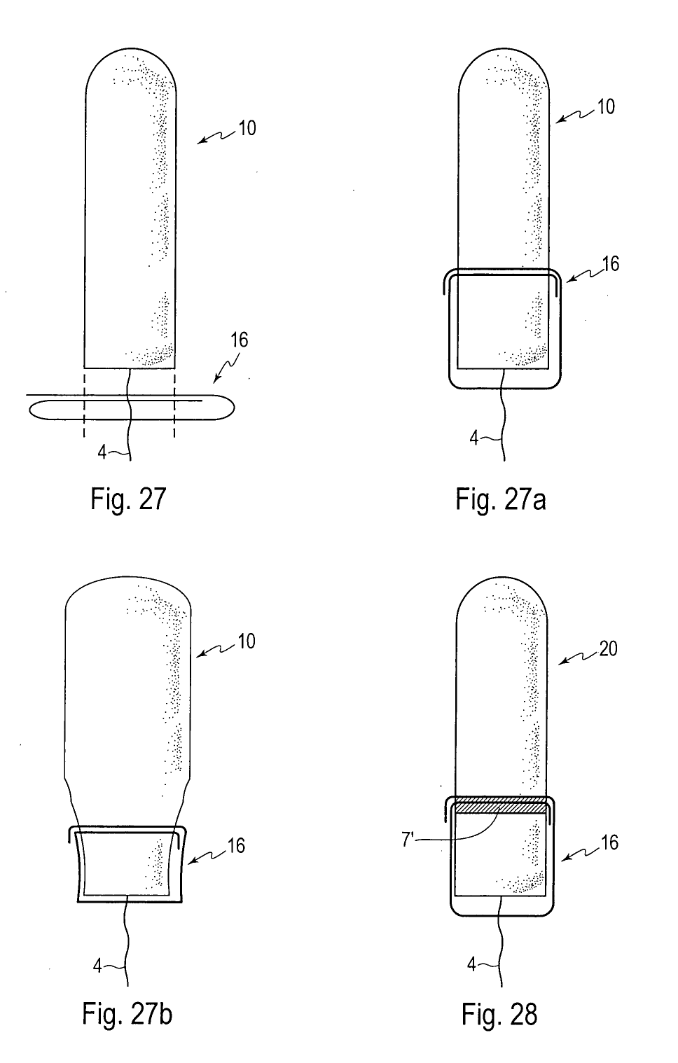

As is shown in FIG. 27, the restrictor band 16 is folded in such a way as to align the orifices 18, with a view to then being positioned around the tampon 10, either via the top or via the bottom of the tampon 10, by inserting the body of the tampon 10 through the orifices 18. In FIG. 27a, this restrictor band 16 is thus introduced via the bottom of the tampon 10.

Advantageously, and as is shown in particular in FIG. 27a, the restrictor band 16 is preferably long enough to leave enough “slack” such that the tampon 10 can dilate. After use, and as is shown in FIG. 27b, the restrictor band 16 will enclose the tampon 10 at the bottom part. In this embodiment, reduced or no friction occurs in the area of the vaginal wall during spasms. Indeed, the slight movement of translation of the tampon due to each spasm does not cause any movement of the restrictor band 16 itself because of the “slack,” which allows the restrictor band 16 to remain motionless in relation to the vaginal wall. Furthermore, withdrawal of the tampon is easier because it happens schematically in two steps due to the “slack.” In other words, the “slack” in the restrictor band 16 allows some freedom of movement of the body of the tampon with respect to the restrictor band 16. Thus, when withdrawing the device, the body of the tampon will move first until it has been moved a sufficient distance that the “slack” in the restrictor band 16 is taken up, whereby the movement of the body of the tampon exerts enough tension on the restrictor band 16 that the body of the tampon and the restrictor band 16 move relative to the walls of the vagina at an equal rate. Also, the pulling force on the body of the tampon elongates the tampon in the axial direction, which decreases the radial diameter of the body of the tampon. As a result, the body of the tampon exerts less radial force on the restrictor band 16, which reduces the frictional force between the walls of the vagina and the restrictor band 16. Accordingly, the longitudinal forces that must be applied to the tampon to withdraw the device are lower than those required with a traditional tampon.

For still greater efficacy, it must be clearly understood that the limitation of the expansion and/or of the absorption of one or more zones of a tampon 20 according to the invention is able to be obtained by simultaneous use, as shown in FIG. 28, of a fluidic barrier 7′, formed with the aid of a hydrophobic substance 7, and also of a restriction member, such as a restrictor band 16. The fluidic barrier 7′ and the zone formed by a restriction member may be located in a proximate area of the tampon, and may be located at substantially the same distance from the proximal end of the tampon.

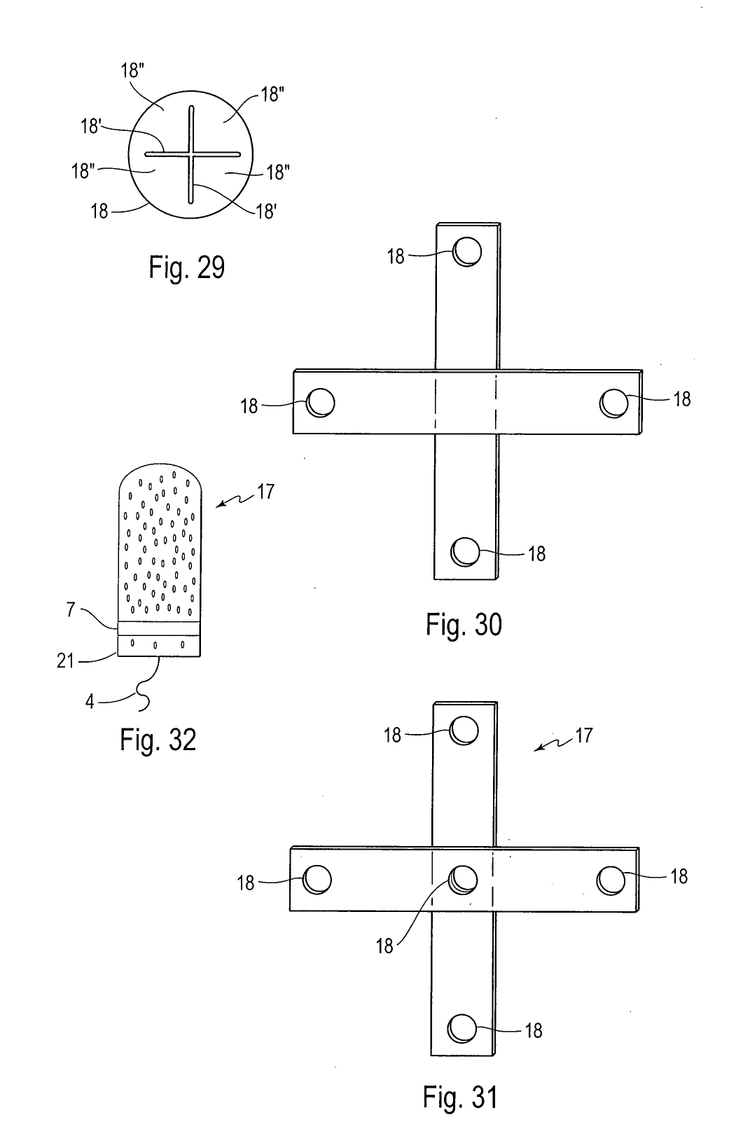

As is shown in FIG. 29, each orifice 18 of the restrictor band 16 may be formed by making two slits 18′ perpendicular to each other, the effect of which is to delimit four substantially triangular flaps 18″ which is oriented upward or downward depending on whether the orifice 18 is introduced via the top or via the bottom of the tampon 10. However, the orifice may be formed by two slits that are not perpendicular to each other, or by more than two slits. A restrictor band 16 may be secured to the tampon by affixing one or more of the triangular flaps 18″ to the tampon by hot pressing, hot melting, sewing, or any other method.

Of course, the material used to form the restrictor band 16 is chosen to be able to resist the pressure of the tampon 10, 20 in the area of the orifices 18.

In the case where the restriction member has the form of a cross 17, each of the four ends comprises an orifice 18, as is illustrated in FIG. 30. If it is a restriction member for the lower part of the tampon 10, a supplementary central orifice 18 is formed for passage of the withdrawal thread 4, as shown in FIG. 31.

It must also be clearly understood that this cross 17 may alternatively be produced with the aid of two separate restrictor bands 16 that are superposed in the area of their central parts and are arranged perpendicular to each other. In the case where this cross 17 is arranged in the lower part of the tampon, the central orifices 18 of the two restrictor bands 16 are superposed in such a way as to permit passage of the withdrawal thread 4.

The restriction member, whether in the form of a restrictor band 16 or a cross 17, is designed to provide some “slack,” such that it is able to adapt to the geometry of the body once it has developed in shape under the effect of the inflation associated with the absorption of the fluid, as is illustrated in particular in FIGS. 24 and 24a. For this reason, the tampon 10 is able to deploy fully in the upper part without being impeded by the restrictor band 16 or cross 17 arranged around it.

Generally speaking, increasing the number of orifices 18 makes it possible to reduce the stresses that have to be withstood by each orifice 18.

In embodiments, the tampon comprises a saturation indicator at its proximal end. A saturation indicator is understood as a means allowing the user to verify the state of saturation or nonsaturation of the tampon by simple contact.

As is illustrated in FIG. 32, the proximal zone 21 situated at the proximal end of the tampon 1 comprises at least one zone in which the expansion and/or the absorption are limited, this zone being formed by partial or total impregnation of the absorbent material of the tampon 1 with a solid hydrophobic substance 7 whose melting point is greater than 37° C. This proximal zone 21 may then serve as a saturation indicator zone.

In fact, because of the presence of the zone in which the expansion and/or the absorption are limited, the proximal zone 21 is soaked only when the tampon 1 is saturated. This proximal zone 21 may thus be used as a saturation indicator. If the proximal zone 21 is neither soaked nor inflated then this indicates to the user that the tampon 1 is not saturated.

In embodiments, the tampon 10 illustrated in FIG. 17 is notable in that the saturation indicator is formed by the proximal zone 22 situated at the proximal end of the tampon 10 comprising at least one zone in which the expansion and/or the absorption are limited by placement of at least one restriction member 11, for example in the form of a ring, string, collar or one or more restrictor bands encircling the zone of the body of the tampon and allowing it to be maintained at this location in its initial shape when the body of the tampon inflates under the effect of the absorption.

Although the invention has been described in connection with particular illustrative embodiments, it will be clear that it is not in any way limited to these embodiments and that it covers all the technical equivalents of the means described, and their combinations, insofar as these come within the scope of the invention.

What is claimed is:

1. A catamenial or sanitary tampon, comprising:
a body comprising an absorbent material, the body having at least one zone containing a solid hydrophobic substance that has a melting point that is greater than 37° C. and that at least partially impregnates the absorbent material of the tampon,
wherein the solid hydrophobic substance limits expansion and/or absorption by the zone when the body of the tampon contacts an absorbable fluid.

2. The tampon according to claim 1, further comprising a withdrawal thread that is at least partially impregnated with a solid hydrophobic substance having a melting point that is greater than 37° C.

3. The tampon according to claim 1, wherein the hydrophobic substance is selected from the group consisting of hydrogenated petrolatum, nonhydrogenated petrolatum, paraffin, and stearin.

4. The tampon according to claim 1, wherein the hydrophobic substance is selected from the group consisting of hydrogenated oils, nonhydrogenated oils, hydrogenated fats, and nonhydrogenated fats.

5. The tampon according to claim 1, wherein the hydrophobic substance is selected from the group consisting of natural and synthetic waxes.

6. The tampon according to claim 1, wherein the hydrophobic substance is hydrogenated petrolatum.

7. The tampon according to claim 1, wherein the hydrophobic substance is beeswax or carnauba wax.

8. The tampon according to claim 1, wherein the hydrophobic substance extends transversely across substantially an entire cross-section of the tampon.

9. The tampon according to claim 1, wherein the at least one zone containing the solid hydrophobic substance is situated at a proximal end of the tampon.

10. The tampon according to claim 1, wherein the at least one zone containing the solid hydrophobic substance is situated at a distance of more than 0.5 mm from a proximal end of the tampon.

11. The tampon according to claim 1, wherein the at least one zone containing the solid hydrophobic substance forms a spiral inside and outside the tampon.

12. The tampon according to claim 1, further comprising another zone containing a solid hydrophobic substance, situated about half way between the two ends of the tampon.

13. The tampon according to claim 1, further comprising an impregnation zone in the form of a funnel configured to channel flow toward a proximal end of the tampon, wherein the occurrence of peripheral leaks due to flow passing to a zone of contact between the walls of a vagina and the tampon is decreased.

14. The tampon according to claim 1, further comprising an impregnation zone in the form of an upturned funnel configured to divert flow from a center of the tampon, wherein accumulation of fluid at a site of fixation of a withdrawal thread is decreased, and wherein occurrence of leaks due to the presence of a withdrawal thread is decreased.

15. The tampon according to claim 1, wherein the solid hydrophobic substance has a melting point that is in a range of 45° C. to 120° C.

16. The tampon according to claim 1, wherein the solid hydrophobic substance has a melting point that is in a range of 68° C. to 100° C.

17. A catamenial or sanitary tampon, comprising:
a body comprising an absorbent material, the body having at least one zone in which expansion and/or absorption are limited, the zone defined by at least one restriction member encircling a portion of the body of the tampon, wherein the restriction member is configured to permanently restrict a diameter of the body of the tampon at said zone from expanding upon absorption of a fluid by the tampon.

18. The tampon according to claim 17, wherein the restriction member comprises at least one member selected from the group consisting of a ring, a washer, a string, a collar, and a foldable restrictor band.

19. The tampon according to claim 17, wherein an outer surface of the restriction member comprises at least one smooth zone configured to reduce friction between the tampon and a vagina during removal of the tampon.

20. The tampon according to claim 17, wherein the restriction member has a spiral shape.

21. The tampon according to claim 18, wherein the restriction member comprises a ring that comprises a separable portion of a wrapper on the tampon.

22. The tampon according to claim 18, wherein the restriction member comprises at least one restrictor band having two ends, and said restrictor band comprises at least one orifice near each of the two ends of the restrictor band, the at least one said orifice having a diameter that is substantially equal to a diameter of the tampon when the tampon is compressed.

23. The tampon according to claim 22, wherein the restrictor band further comprises at least a third orifice in a central part of the restrictor band for passage of a withdrawal thread.

24. The tampon according to claim 22, wherein at least one said orifice in the restrictor band is defined by a plurality of slits.

25. The tampon according to claim 24, wherein at least one said orifice in the restrictor band is defined by two slits perpendicular to each other, in such a way as to delimit four flaps.

26. The tampon according to claim 25, wherein at least one of the four flaps is affixed to the body of the tampon.

27. The tampon according to claim 18, wherein the tampon comprises at least two superposed restrictor bands arranged perpendicular to each other at a location at which the restrictor bands are superposed.

28. The tampon according to claim 18, wherein the restriction member comprises at least one restrictor band, and the restrictor band is configured to have a length that permits expansion of the tampon body outside said zone when the tampon body is placed in contact with a fluid that is to be absorbed.

29. The tampon according to claim 18, wherein the restriction member comprises at least one washer having an orifice that is defined by a plurality of slits.

30. The tampon according to claim 29, wherein the restriction member comprises at least one washer having an orifice that is defined by two slits perpendicular to each other in such a way as to delimit four flaps.

31. The tampon according to claim 30, wherein at least one of the four flaps is affixed to the body of the tampon.

32. The tampon according to claim 30, wherein two opposite flaps each comprise a hole permitting passage of a withdrawal thread.

33. The tampon according to claim 17, further comprising a fluidic barrier comprising a hydrophobic substance.

34. The tampon according to claim 33, wherein the fluidic barrier and the zone defined by the at least one restriction member are located in a proximal area of the tampon.

35. The tampon according to claim 34, wherein the fluidic barrier and the zone defined by the at least one restriction member are located at substantially the same distance from a proximal end of the tampon.

36. The tampon according to claim 17, wherein the restriction member comprises a withdrawal thread.

37. The tampon according to claim 17, further comprising a withdrawal thread attached to the restriction member.

38. A strip of absorbent material comprising at least one zone in which the absorbent material is partially or totally impregnated with a solid hydrophobic substance having a melting point that is greater than 37° C., wherein said strip of absorbent material is configured to be formed into a body of a tampon.

39. The strip of absorbent material according to claim 38, wherein the hydrophobic substance is selected from the group consisting of hydrogenated and nonhydrogenated oils and fats.

40. The strip of absorbent material according to claim 38, wherein the hydrophobic substance is selected from the group consisting of natural and synthetic waxes.

41. The strip of absorbent material according to claim 38, wherein the hydrophobic substance is hydrogenated petrolatum.

42. The strip of absorbent material according to claim 38, wherein the hydrophobic substance is beeswax or carnauba wax.

43. A method for manufacturing a catamenial or sanitary tampon, comprising:
injecting a solid hydrophobic substance having a melting point that is greater than 37° C. into a thickness of a strip of absorbent material, and forming said strip of absorbent material into a body of a tampon.

44. The method of claim 43, wherein the hydrophobic substance is injected discontinuously.

45. The method of claim 43, further comprising a step of folding and optionally of compressing the strip of absorbent material, followed by a step of heating said strip of absorbent material to a temperature above a melting point of the hydrophobic substance, thereby binding together two or more portions of said strip containing said hydrophobic substance.

46. The method of claim 43, wherein the hydrophobic substance is selected from the group consisting of hydrogenated and nonhydrogenated oils and fats.

47. The method of claim 43, wherein the hydrophobic substance is selected from the group consisting of natural and synthetic waxes.

48. The method of claim 43, wherein the hydrophobic substance is hydrogenated petrolatum.

49. The method of claim 43, wherein the hydrophobic substance is beeswax or carnauba wax.

50. A tampon, comprising:
a body comprising an absorbent material, and
a saturation indicator at a proximal end of the tampon.

51. The tampon according to claim 50, wherein the indicator is defined by a proximal zone situated at said proximal end of the tampon, the proximal zone comprising at least one zone in which expansion and/or absorption are limited, the zone containing a solid hydrophobic substance that has a melting point that is greater than 37° C. and that at least partially impregnates the absorbent material of the tampon.

52. The tampon according to claim 50, wherein the indicator is defined by a proximal zone situated at said proximal end of the tampon, the proximal zone comprising at least one zone in which expansion and/or absorption are limited, the zone defined by at least one restriction member encircling the tampon, wherein the restriction member is configured to permanently restrict a diameter of the body of the tampon at said zone when the tampon expands upon absorption of a fluid.

53. The tampon according to claim 52, wherein the restriction member comprises at least one member selected from the group consisting of a ring, a washer, a string, a collar, and a restrictor band.

54. The tampon according to claim 52, wherein an outer face of the restriction member comprises at least one smooth zone configured to reduce friction between the tampon and a vagina during removal of the tampon.

55. The tampon according to claim 52, wherein the restriction member has a spiral shape.

56. The tampon according to claim 52, wherein the restriction member is a ring that comprises a separable portion of a wrapper on the tampon.

57. The tampon according to claim 53, wherein the restriction member comprises at least one restrictor band having two ends, and each said restrictor band comprises at least one orifice near each of the two ends of the restrictor band, at least one said orifice having a diameter that is substantially equal to a diameter of the tampon when the tampon is compressed.

58. The tampon according to claim 57, wherein the restrictor band further comprises at least a third orifice in a central part of the restrictor band, the third orifice configured for passage of a withdrawal thread.

59. The tampon according to claim 57, wherein at least one said orifice in the restrictor band is defined by a plurality of slits.

60. The tampon according to claim 59, wherein at least one said orifice in the restrictor band is defined by two slits perpendicular to each other, in such a way as to delimit four flaps.

61. The tampon according to claim 53, wherein the restriction member comprises at least two superposed restrictor bands arranged perpendicular to each other at a location at which the restrictor bands are superposed.

62. The tampon according to claim 53, wherein the restrictor band is configured to have a length that permits expansion of the tampon body outside said zone when the tampon body is placed in contact with a fluid that is to be absorbed.

63. The tampon according to claim 53, wherein the restriction member comprises at least one washer having an orifice that is defined by a plurality of slits.

64. The tampon according to claim 63, wherein the restriction member comprises at least one washer having an orifice that is defined by two slits perpendicular to each other in such a way as to delimit four flaps.

65. The tampon according to claim 64, wherein two opposite said flaps each comprise a hole permitting passage of a withdrawal thread.

66. The tampon according to claim 52, wherein the restriction member comprises a withdrawal thread.

67. The tampon according to claim 52, further comprising a withdrawal thread attached to the restriction member.

68. A tampon, comprising:
a body comprising an absorbent material,
a layer of a hydrophobic substance extending across a circumference of the body of the tampon, wherein the hydrophobic substance at least partially impregnates the absorbent material.

69. The tampon according to claim 68, further comprising a ring of material encircling said circumference of the body of the tampon at a location of the layer of the hydrophobic substance.
